(19) United States
(12) Patent Application Publication
(10) Pub. No.: US 2021/0031021 A1
(43) Pub. Date: Feb. 4, 2021

(54) MEDICAL TUBE

(71) Applicant: Synecco Limited, Galway (IE)

(72) Inventors: Brian Ledwith, Galway (IE); Mark Costello, Mayo (IE)

(21) Appl. No.: 16/526,661

(22) Filed: Jul. 30, 2019

(51) Int. Cl. A61M 39/10; A61J 15/00; A61M 25/00

(52) U.S. Cl. CPC A61M 39/1011; A61J 15/0003; B29C 45/14336; A61M 25/0014; A61J 15/0076

(57) ABSTRACT

Aspects of the present invention relate to a medical tube for delivering nutrients or medication to a patient. The tube comprises a flexible tubular body for conveying the nutrients or medication to the patient and a small-bore connector for connecting the medical tube to a syringe or pump. The small-bore connector is secured to the tubular body by an overmoulded component that encapsulates a portion of the tubular body and a portion of the connector thereby securing the connector relative to the tubular body of the medical tube.

### TECHNICAL FIELD

The present disclosure relates to a method of securing a relatively rigid connector to a relatively flexible tubular body to form a medical tube for use in various medical applications. Aspects of the invention relate to a medical tube for delivering, for example but not limited to, nutrients or medication to a patient, to an enteral feeding tube for enteral feeding, and to a method of attaching a small-bore connector to a medical tube.

### BACKGROUND

When patients have problems eating or digesting solid foods it is sometimes necessary to deliver liquid food to the patient's stomach via an enteral feeding tube. The enteral feeding tube may be inserted up a patient's nose and down the patient's oesophagus such that a distal end of the tube is positioned within the patient's stomach. Food can be delivered to the patient's stomach by connecting a syringe or pump to a proximal end of the enteral feeding tube via a connector and passing food through the tube directly to the patient's stomach.

The inner linings of a patient's stomach, oesophagus and nose are delicate and care should be taken to avoid damaging the gastric lining or causing discomfort to the patient when enteral feeding. As such, it is desirable that the enteral feeding tube is made from a soft, highly flexible plastics material in order to minimise discomfort to the patient. Furthermore, the soft plastics material minimises the risk of gastric wall punctures and/or soft tissue damage that could be caused by tubes made of harder plastics materials.

A problem with enteral feeding tubes historically was that the connector used to connect the syringe or pump to the tube was not unique to enteral feeding. In particular, various medical lines and tubes used a universal luer lock connector for compatibility with a variety of different medical devices. Thus, it was possible for a healthcare professional to connect the feeding tube accidentally to non-enteral devices such as IV lines, urinary catheters or ventilator tubing, which could cause injury to the patient.

This problem of misconnection is compounded by the fact that patients often have multiple lines or medical tubes connected to them when they are in hospital to deliver the appropriate medication, gases and nutrients. The large number of lines or tubes increases the likelihood of misconnection.

To mitigate the risk of misconnection, the healthcare industry standardised small-bore connectors such that misconnection between various medical devices was not possible. As part of this transition, a new ISO standard was introduced. ISO 80369 was developed to improve patient safety and to reduce the risk of small-bore misconnections in liquid and gas healthcare applications.

In the context of the invention as it will be understood by the skilled reader, the term ‘small-bore connector’ relates to non-luer connectors that are unique to the medical application for which they are used such that misconnection is prevented. Examples of small-bore connectors are outlined in the ISO 80369 series. A small-bore connector is defined as having an inner diameter of less than 8.5 mm, whereas a luer lock connector is defined by the ISO 594-1 and ISO 594-2 standards as “a conical fitting with a 6% taper for syringes, needles and certain other medical equipment.”

As part of this series, ISO 80369-3 was introduced to prevent misconnection of connectors for enteral feeding tubes. In the case of enteral feeding, the connector was standardised to an ENFit® connector that is only compatible with other enteral feeding devices. The ISO 80369-3 standard specified the shape, dimensions and flexural modulus of the enteral connector, namely the shape of the ENFit® connector and also the functional performance of the connector. The ENFit® connector may be made from a plastics material such as polyurethane resin material or ABS with a flexular modulus in excess of 700 MPa, which makes the connector much harder or more rigid than the previous universal luer lock connector which typically had a flexural modulus of about 40 MPa.

Connecting the relatively soft enteral feeding tube to the new, relatively hard, ENFit® connector presents a number of challenges in terms of achieving a secure and robust connection between the relatively hard connector hub and the relatively soft enteral feeding tube.

Prior to the introduction of the ISO 80369-3 standard it was known to overmould a relatively soft push-fit connector, made from for example, a soft plastics material with a flexural modulus of about 40 MPa, to the enteral feeding tube. FIG. 1 shows a connector 10 directly overmoulded to an enteral feeding tube 12. During manufacture, the enteral feeding tube 12 is positioned within a mould and the material of the connector 10 is injected into the mould such that the connector 10 is formed and overmoulded onto the feeding tube 12 thereby both forming, sealing and securing the connector 10 to the feeding tube 12 in a single step.

This process worked well for the universal luer lock connector as the Vicat softening temperature (VST) of the connector 10 is about 106° C. which is lower than the melting temperature of the tube 12. As such, the energy required to fill the mould cavity in the overmoulding process is relatively low meaning the temperature of the enteral feeding tube 12 did not exceed its melting or softening temperature. Of course, it is undesirable to melt or over-soften the enteral feeding tube 12 during manufacture as this may cause the tube 12 to deform unpredictably and potentially to collapse inwardly, restricting or blocking its lumen, or resulting in leaks between the connector and tube.

The melting temperature of the ENFit® connector is higher than the melting temperature of the universal luer lock connector. As such the aforementioned method of overmoulding a connector to an enteral feeding tube 12 is not suitable because the temperatures required to overmould the ENFit® connector would melt or at least over-soften the relatively soft enteral feeding tube 12. If the material of the tube 12 is changed to increase its melting or softening temperature then typically the hardness or rigidity of the material will also increase. As noted above, this is undesirable for the application of enteral feeding as a hard tube would cause discomfort to the patient, be more difficult to insert and also risk a gastric puncture or soft tissue damage.

Another challenge is that the additives used in the material of the ENFit® connector may leach out of the connector and form as an oily film over the outer surface of the connector. This prevents conventional adhesives from being able to bond the connector securely and reliably to the body of the tube.

It is also known to use a slip fit to connect a tube body to a small-bore connector. For example, the connector may comprise a male formation onto which the medical tube may be pushed axially. However, the interference between the connector and the tube required to achieve the 15N removal force specified by the intravascular catheter ISO 10555 requirements and the non-intravascular EN1618 requirements, is too high for manual insertion. Furthermore, the interference requirements of the slip fit connection can cause stress cracking in the tube body, which unduly limits the life of the enteral feeding tube.

As such, there is a need for a new method of securing a small-bore connector to a tube for use in a medical application that complies with the requirements of the ISO-80369 series standard.

It is an aim of the present invention to address one or more of the disadvantages associated with the prior art.

### SUMMARY OF THE INVENTION

Aspects and embodiments of the invention provide a medical tube for delivering fluids to a patient, an enteral feeding tube for enteral feeding, and a method of attaching a small-bore connector to a medical tube for use in a medical application.

In a broad sense there is provided apparatus for delivering a fluid such as nutrients or medication to a patient. The apparatus comprises: a medical tube for conveying the nutrients or medication to the patient and a small-bore connector for connecting the tube to a syringe or pump. The connector is secured to the tube by an overmoulded component that encapsulates a portion of the tube and a portion of the connector thereby securing the connector relative to the feeding tube.

According to an aspect of the present invention there is provided a medical tube for delivering fluids to a patient, the tube comprising: a flexible tubular body for conveying the fluids; and a connector for connecting the medical tube to a source of fluids; wherein the connector is secured to the tubular body by an overmould structure that encapsulates a portion of the tubular body and at least a portion of the connector thereby securing the connector to the tubular body.

This is advantageous as the overmould structure allows two components that are not suitable for standard overmoulding or bonding to be connected. For example, in the application of enteral feeding it allows a relatively hard connector to be securely connected to the medical tube or enteral feeding tube. The overmould structure encapsulates a portion of the tube and at least a portion of the connector which beneficially provides a strong and robust connection between the two otherwise incompatible components.

Furthermore, the overmould structure forms a seal between the connector and the tube. This is beneficial as it prevents fluids being carried by the medical tube from leaking at the point that the connector joins the tubular body.

The skilled reader will understand that the fluids may be a liquid or gas. For example, the fluids may be liquid nutrients, liquid medication, air, oxygen or any other fluid that may be used in a medical application. The medical tube may be an enteral feeding tube for enteral feeding. Furthermore, the connector may be a small-bore connector suitable for use in various medical applications.

In one embodiment the connector may comprise a central bore and wherein a distal end of the tubular body may be received within the central bore. The tube may be partially received within the central bore such that the central bore provides a strain relief for the tube. This is advantageous as placing part of the tube within the central bore of the connector compensates for any strain on the tube that otherwise may cause the connector to disconnect from the tube.

In another embodiment the connector may comprise a mechanical retention feature for securing the overmould structure to the connector. The mechanical connection feature may create an additional mechanical connection or mechanical retention between the overmoulded component and the connector. The mechanical connection feature may be a barb or a barbed feature, for example a proximally-tapering barb. Beneficially, the overmoulded component may mould or conform to the shape of the barbed feature such that when the overmoulded component sets it mechanically engages the barbed feature. The mechanical connection feature may also be a radially extending tab, flange or ridge that is configure to engage and retain the overmould structure.

The mechanical retention feature may comprise a distally-facing wall and a proximally-facing wall that are mutually spaced in a longitudinal direction to define an annular recess between them. In one embodiment a portion of the overmould structure may extend into and engage within the annular recess. This is beneficial as it further strengthens the connection between the connector and the tube. The portion of the overmould structure located within the annular recess inhibits and resists movement of the overmould structure in a longitudinal direction. In an embodiment the overmould structure may encapsulate the mechanical retention feature.

In one embodiment the overmould structure may taper from a first diameter at a distal end to a second diameter at a proximal end. This is beneficial as the generally tapering profile of the overmould structure reduces the material requirement. Furthermore, the tapering profile is easy to handle and grip thereby making the connector easy to use for a healthcare professional. The generally tapered profile beneficially minimises the material requirements of the overmould structure thereby minimising the energy requirements for overmoulding. This is beneficial as reducing the energy requirements also reduces the temperature within the mould.

In an embodiment the taper may be longitudinally aligned with the mechanical retention feature. This is beneficial as it allows the overmould structure to have a greater thickness in the region of the mechanical retention feature thereby reducing the risk of stress cracking. Furthermore, the reduced thickness in the region of the tubular body beneficially reduces the material requirements of the overmould structure.

The overmould structure may extend proximally from the connector to a length that exceeds the first diameter. This is beneficial as the overmould structure extends along a portion of the tubular body thereby increasing the surface area of the tube that the overmould structure may bond or adhere to. Furthermore, the overmould structure provides support to the tubular body and as such extending the overmould structure along a portion of the tubular body supports the tubular body, provides strain relief and provides support against lateral movement.

In one embodiment the outer surface of the overmould structure is aligned with a longitudinal axis of the connector at opposing ends of the taper. This creates a generally stepped outer profile on the overmould structure. The overmould structure may comprise a further taper at a proximal end of the overmould structure. At least one of the tapers may be frusto-conical.

In another embodiment the overmould structure may be bonded to the connector and to the tubular body. This is beneficial as bonding the overmould structure to a portion of the hub or the connector secures the overmould structure to the hub and the connector, thereby securing the connector to the hub. The bond between the overmould structure and the connector and tube may be a heat-bond formed as the overmoulding component cures and solidifies following the overmoulding process. The heat-bond formed between the connector and the tube improves the seal formed between the connector and the tube. As such a robust seal is formed between the connector and the tube which prevents fluids leaking from the join between the connector and the tube.

In one embodiment the connector may have a flexural modulus of at least 700 MPa and the tubular body may have a flexural modulus of about 100 MPa or less. For example, the tubular modulus may have a flexural modulus of about 40 MPa. In another embodiment the flexural modulus of the overmould structure may be greater than or equal to the flexural modulus of the tubular body and/or it may be less than or equal to the flexural modulus of the connector.

In another embodiment the overmould structure is moulded from an injection-mouldable version of the material of the tubular body. The material of the overmould structure may be the same material as the material of the tube or it may be another plastics material. The overmould structure may have a melting or softening temperature that is equal to or lower than a melting or softening temperature of the tubular body. The overmould structure may have a melting or softening temperature that is equal to or higher than a melting or softening temperature of the tubular body.

According to another aspect of the present invention there is provided a feeding tube for enteral feeding, the feeding tube comprising: a flexible tubular body for conveying nutrients to a patient's stomach; and a connector for connecting the flexible tubular body to a source of nutrients; wherein the connector is secured to the tubular body by an overmould structure that encapsulates a portion of the tubular body and at least a portion of the connector thereby securing the connector to the tubular body.

In an embodiment the apparatus for enteral feeding may further comprise a syringe or a pump connected to the connector.

According to a yet further aspect of the present invention there is provided a method of manufacturing a medical tube for use in a medical application, the medical tube comprising a flexible tubular body and a connector, the method comprising: placing an end portion of the tubular body inside the connector; placing the tubular body and the connector into a mould; and injecting an overmould material into the mould to encapsulate a portion of the tubular body and at least a portion of the connector, forming an overmould structure that secures the connector relative to the tubular body.

The skilled reader will appreciate that the step of injecting an overmould material may not be carried out via conventional overmoulding and may encompass alternative manufacturing techniques such as vacuum casting, compression moulding or from injecting a two-part resin such as epoxy.

In one embodiment the method may comprise moulding the connector prior to overmoulding. As such the method of manufacturing the medical tube is a multiple step process in which the connector is moulded prior to being joined to the tubular body.

Within the scope of this application it is expressly intended that the various aspects, embodiments, examples and alternatives set out in the preceding paragraphs, in the and/or in the following description and drawings, and in particular the individual features thereof, may be taken independently or in any combination.

### BRIEF DESCRIPTION OF THE DRAWINGS

One or more embodiments of the invention will now be described, by way of example only, with reference to the accompanying drawings, in which:

FIG. 1 is a schematic side view of a prior art connector and feeding tube;

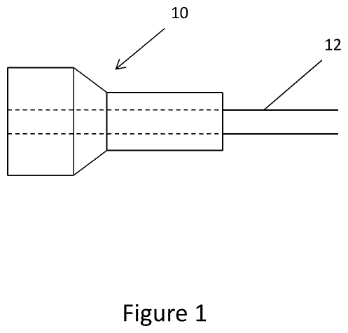

FIG. 2 is a perspective view of a connector and a feeding tube of the invention;

FIG. 3 corresponds to FIG. 2 but shows the connector and feeding tube joined by an overmoulded component;

FIG. 4 is a cross-sectional view through the connector, feeding tube and overmoulded component of FIG. 3;

FIG. 5 is a flow diagram outlining the method steps of the overmoulding process;

FIG. 6 is a perspective view of the connector and tube of FIG. 2 positioned within an open mould;

FIG. 7 is a perspective view of the mould of FIG. 6, when closed and ready for injection moulding of the overmoulded component;

FIG. 8 corresponds to FIG. 6 but shows the connector and tube positioned within the re-opened mould after the overmoulded component has been formed; and

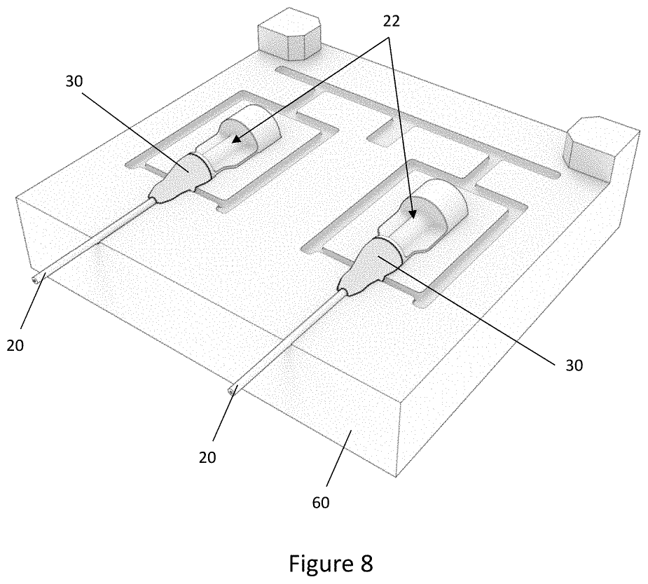

FIG. 9 is a cross-sectional view through the connector, feeding tube and overmould of an alternative embodiment of the invention.

### DETAILED DESCRIPTION

In general terms, embodiments of the invention relate to a method of securing a small-bore connector to a tubular body or line to make a medical tube for conveying fluid such as medication, nutrients, air or oxygen to a patient. In a broad sense, the method comprises securing the connector to the tube by overmoulding a plastics material over at least a portion of the connector and a portion of the tube. The overmoulded component may, at least partially, encapsulate a portion of the connector and a portion of the tube to secure the connector relative to the tube.

The skilled reader will understand that whilst the invention is described herein in the context of enteral feeding, the inventive concepts described may be applied to other medical applications; for example, but not limited to: breathing systems, urinary collection systems, limb cuff inflation, neuraxial applications and intravenous systems.

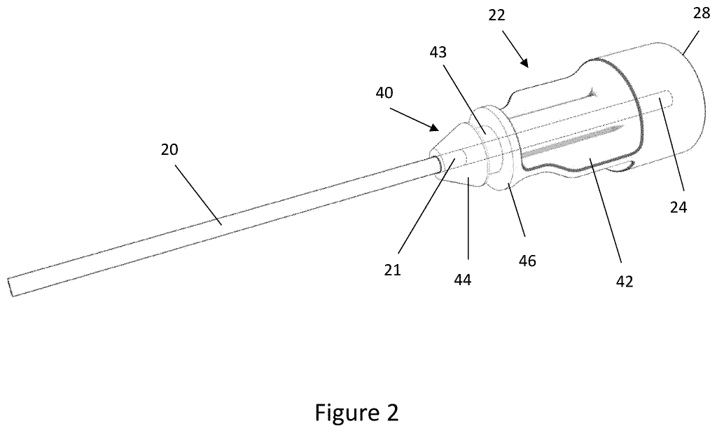

To place embodiments of the invention in a suitable context, reference will firstly be made to FIG. 2 which shows an enteral feeding tube 20 and a hub or connector 22 assembled but not yet fully secured together. The enteral feeding tube 20 comprises medical tubing that may be inserted up a patient's nose and down the oesophagus such that nutrients may be delivered to the stomach. The enteral feeding tube 20 therefore comprises a highly-flexible tubular body. The tubular body is made from a soft plastics material, preferably an aliphatic compound. An example of such a material is a soft polyurethane with a flexural modulus of about 40 MPa or less. Aliphatic compounds are single-bond structures that are soft and thus suitable for insertion into a patient's stomach in the form of an enteral feeding tube. The skilled reader will understand that other tube materials may be used and that the tubular body may equally be made from an aromatic polyurethane.

The connector 22 of FIG. 2 is exemplified here as an ENFit® connector although the skilled reader will appreciate that the connector type will vary depending on the medical application. To comply with the ISO-80369 series standard the connector 22 is made from a plastics material with a flexural modulus in excess of 700 MPa. For example, the connector 22 may be moulded from a polyurethane resin for medical applications. As the connector 22 is relatively hard compared to the soft plastics tube 20, the melting or softening temperature of the connector 22 is higher than the melting or softening temperature of the tube 20. As such it is not possible to overmould the connector 22 on to the tube 20 using a standard overmoulding process as the energy required to do so would cause the tube 20 to melt or over-soften and hence deform unacceptably. This problem is particularly relevant to situations where a relatively hard connector, for example with a flexural modulus in excess of 700 MPa is to be connected to a relatively soft and/or thin walled tube with a flexural modulus of less than 100 MPa.

The connector 22 comprises an interface 28 at its distal end that is unique to the medical application for which the connector 22 is designed. In the context of enteral feeding, the interface 28 comprises a threaded interface for connecting the connector 22 to an enteral syringe or pump (not shown) to deliver liquid food to the patient's stomach. The interface 28 is unique to the medical application to prevent misconnection of the connector 22 to the wrong syringe or pumping device.

A central bore 24 runs along a central longitudinal axis of the connector 22 such that fluids may be conveyed to the feeding tube 20 from a pump or syringe connected to the connector 22. As shown in FIG. 2, a distal end portion 21 of the tube 20 is inserted into the proximal end of the central bore 24 of the connector 22. There may be a clearance fit between the central bore 24 and the tube 20 to allow an operator to easily position the tube 20 within the central bore 24 or there may be transition fit between the central bore 24 and the tube 20. It is desirable to minimise the gap between the central bore 24 and the tube 20 to prevent the overmould structure 30 from flowing into the gap during the overmoulding process. Optionally, the central bore 24 could have a circumferential shoulder or ridge to limit the depth of insertion of the tube 20 into the proximal end of the connector 22.

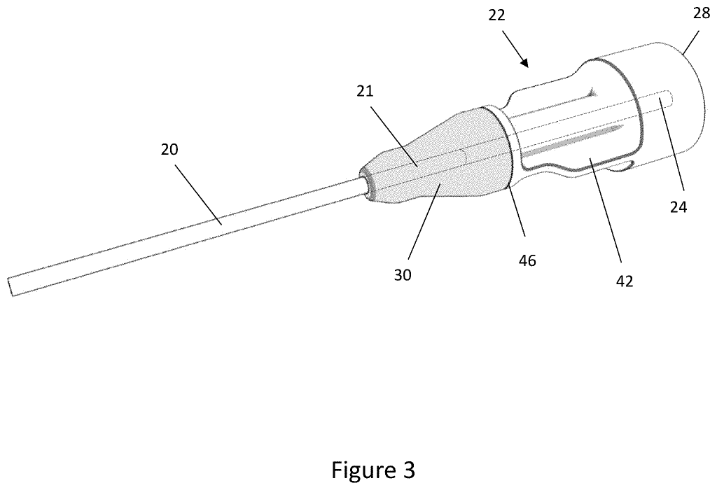

Turning now to FIG. 3, the tube 20 and connector 22 are shown after they have been joined by overmoulding an overmould component or structure 30 over a distal portion of the tube 20 and a proximal portion of the connector 22. The overmould structure 30 comprises a plastics material that at least partially encapsulates the tube 20 and the connector 22 thereby securing the connector 22 to the tube 20. The overmould structure 30 bonds to the tube 20 and connector 22 as the plastics material of the overmould structure 30 cures and sets in the mould 60. The bonds between the overmould structure 30 and the tube 20 and the connector 22 may therefore be heat bonds that are formed as the overmould structure 30 cures in the mould.

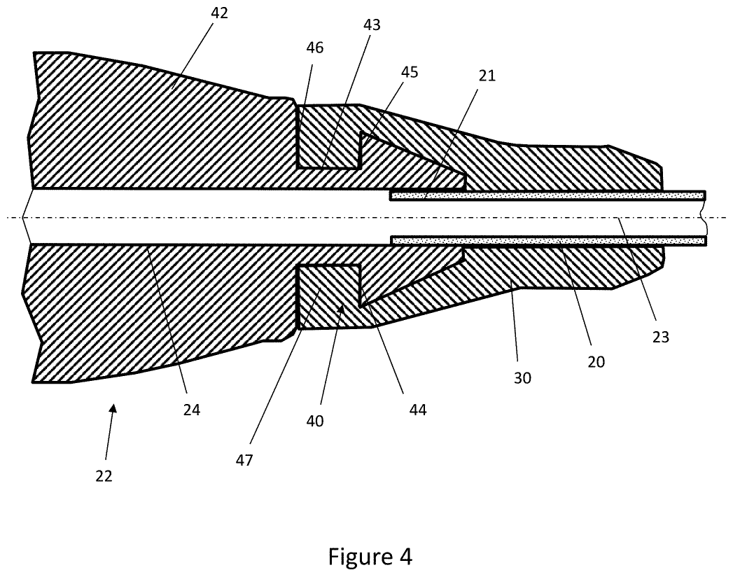

FIG. 4 shows a longitudinal sectional view of the connector 22, the tube 20 and the overmould structure 30, on a plane that contains the central longitudinal axis 23. The overmould structure 30 encases or encapsulates the distal portion of the tube 20 and the proximal portion of the connector 22.

The connector 22 comprises a connector body 42. A barbed portion 40 extends from a proximal end of the connector body 42 and is rotationally symmetrical about the central longitudinal axis 23. The central bore 24 extends along the central longitudinal axis 23 of the connector body 42 to define a passage that extends longitudinally through the full length of the connector body 42 and also through the barbed portion 40 at the proximal end of the connector body 42. Thus, the central bore 24 penetrates the full length of the connector 22.

The connector body 42 comprises a planar proximally-facing wall 46 from which the barbed portion 40 extends. The wall 46 lies in a plane that is orthogonal to the central longitudinal axis 23.

The barbed portion 40 comprises a tubular neck 43 that is integral with and extends longitudinally from the wall 46 in a proximal direction. A hollow frusto-conical barb 44 is positioned at the opposing proximal end of the neck 43 and tapers in a proximal direction away from the neck 43.

The barb 44 comprises a distally-facing wall 45 that faces the proximally-facing wall 46 of the connector body 42 and is spaced from that wall 46 by the length of the neck 43. In this example, the wall 45 also lies in a plane that is orthogonal to the central longitudinal axis 23 and so is parallel to the wall 46.

As shown in FIG. 4, the overmoulded structure 30 encapsulates the barbed portion 40 and, in doing so, abuts and adheres to the outer surface of the barbed portion 40, including the proximally-facing wall 46 of the connector body 42 and to the distally-facing wall of the barb 44. This is beneficial as the walls 45, 46, the neck 43 extending between them and the frusto-conical surface of the barb 44 provide a large surface area for the overmoulded structure 30 to heat-bond to the connector 22. This improves the strength of the connection between the overmould structure 30 and the connector 22.

As the overmould structure 30 cures the surface of the connector 22 and tube 20 may melt slightly thereby forming a heat-bond with the overmould structure 30. The heat-bond forms a seal between the overmould structure 30 and the connector 22 and tube 20 which beneficially prevents fluids leaking at the point the tube 20 is connected to the connector 22.

Additionally, the barbed portion 40 is an example of a mechanical retention feature or engagement formation that further improves the strength of the connection between the overmould structure 30 and the connector 22. Specifically, a portion of the overmould structure 30 extends into and fills the annular recess 47 between the distally-facing wall 45 of the barb 44 and the proximally-facing wall 46 of the connector body 42. This portion of the overmould structure 30 trapped between the barb 44 and the connector body 42 inhibits longitudinal movement of the overmould structure 30 relative to the connector 22. Thus, the barbed portion 40 provides an additional mechanical connection between the overmould structure 30 and the connector 22.

As shown in FIG. 4, the overmould structure 30 tapers from a relatively wide distal end that abuts the proximally-facing wall 46 of the connector body 42 to a relatively narrow proximal end that encircles the tube 20. The overmould structure 30 is elongate, extending proximally from the connector body 42 to a length that exceeds the width of the distal end of the overmould structure 30. Overall, the length of the overmould structure 30 parallel to the central longitudinal axis 23 is more than twice the length of the barbed portion 40. Thus, a substantial length of the overmould structure 30 extends proximally beyond the barb 44 to contact the outer surface of the tube 20. This further maximises the surface area of contact between the overmould structure 30 and the tube 20, thereby strengthening the resistance of the tube 20 to proximal pull-out forces and adding strain relief to the assembly.

More specifically, the overmould structure 30 tapers in steps that, moving proximally, are defined by a cylindrical distal end portion, a first frusto-conical step that tapers proximally, a cylindrical intermediate portion and a second frusto-conical step that tapers proximally to the proximal end. The distal end portion and the intermediate portion each have an outer surface that lies parallel to the central longitudinal axis 23.

The distal end portion of the overmould structure 30 is aligned longitudinally with the neck 43. The first frusto-conical step is aligned with, and extends proximally beyond, the tapering surface of the barb 44. The second frusto-conical step reduces the diameter of the overmould structure 30 to near the outer diameter of the tube 20.

By virtue of its tapered and preferably stepped outer shape, the overmould structure 30 generally follows the underlying shape of the barb feature 40 and the tube 20. This reduces the volume of material in the overmould structure and so beneficially reduces material consumption and the energy and pressure requirements for its formation by injection moulding. This beneficially minimises the temperature required in the overmoulding process which in turn prevents the soft plastics material of the tube 20 from melting or over-softening and thus deforming. Furthermore, the increased thickness of the distal end portion and the first frusto-conical step of the overmould structure 30 in the region of the barb feature 40 reduces the risk of the barb 44 inducing stress cracks in the overmould structure 30.

The smooth outer finish provided by the overmould structure is aesthetically pleasing, easy to handle and minimises dirt traps.

The overmould structure 30 is a plastics material with a melting or softening temperature below the melting or softening temperature of the tube 20. Beneficially, this prevents the tube 20 from melting or over-softening during the overmoulding process. The overmould structure 30 may be an injection-mouldable form of the material from which the tube 20 is made. For example, the overmould structure 30 could be moulded from an injection-mouldable form of polyurethane. or the overmould structure 30 could be moulded from another plastics material that is suitable for injection moulding.

The overmould structure 30 is typically harder and more rigid than the flexible tubular body of the tube 20. As such, the overmould structure 30 provides strain-relief support to the tube 20 thereby reducing the effect of lateral loads on the tube 20. This is beneficial as cyclical loading may cause the connection between the connector 22 and the tube 20 to fatigue over time. Furthermore, the relative strength and rigidity of the overmould structure 30 prevents the overmould structure deforming and losing its shape over time. This beneficially ensures that the connection between the connector 22 and the tube 20 is not weakened or compromised over time.

Conversely, the overmould structure 30 is not as rigid as the connector 22 and as such the overmould structure 30 may flex slightly if it is subject to lateral loading. In this sense, the overmould structure 30 can absorb lateral loads. This is beneficial as it reduces the loading that the soft flexible tube 20 is subject to thereby improving the quality of the connection between the connector 22 and the tube 20.

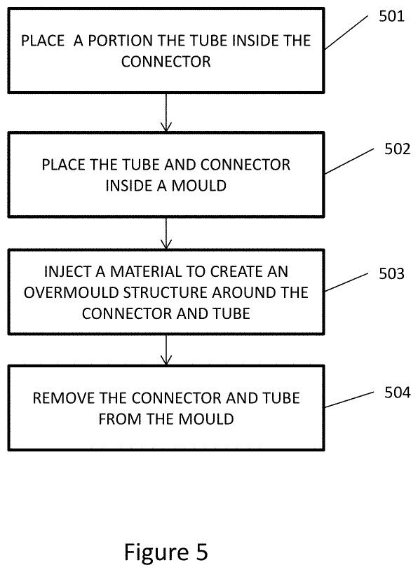

Method steps for securing the connector 22 to the tube 20 with the overmould structure 30 are outlined in the flow chart of FIG. 5. The first step 501 comprises positioning the distal end portion 21 of the tube 20 within the connector 22 as shown in FIG. 2. A desired length of the tube 20 is inserted into the central bore 24 by an operator such that the distal end portion 21 of the tube 20 is fully received within the central bore 24 of the connector 22.

Typically, between about 2 mm and 20 mm of the tube 20 is inserted into the central bore 24 of the connector 22 prior to overmoulding the overmould structure 30. This is beneficial as it provides strain relief that prevents the tube 20 inadvertently being disconnected from the connector 22 in the event that the tube 20 is pulled either longitudinally or laterally.

The connector 22 and the tube 20 may be assembled on a core pin or a mandrel (not shown) prior to being positioned in the mould 60. This is beneficial as it helps to stabilise and secure the connector 22 relative to the tube 20 prior to forming the overmould structure 30.

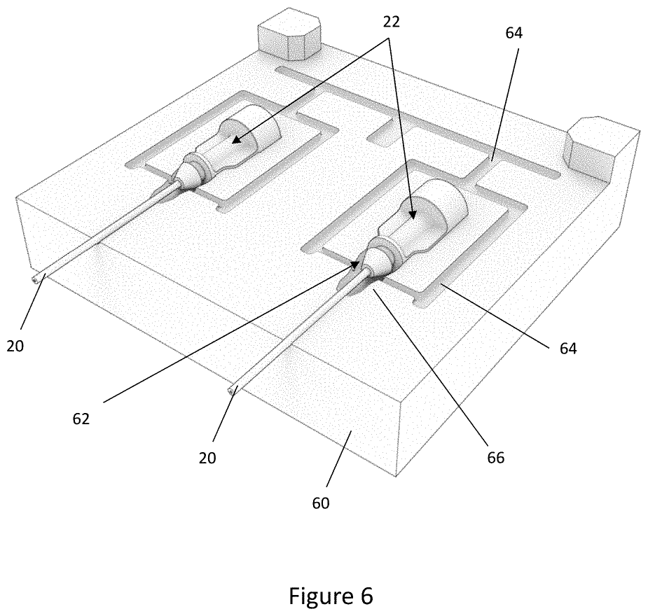

Next, in step 502, the tube 20 and connector 22 are placed within a mould 60 as shown in FIG. 6. The mould 60 comprises at least one bed 62 for receiving the connector 22 and the tube 20. An overmould chamber 66 surrounds the distal portion of the tube 20 and the proximal portion of the connector 22 that are to be encapsulated by the overmould structure 30. Channels 64 in the mould provide a flow path for the overmould material to fill the overmould chamber 66. The tube 20 may be clamped relative to the mould 60 to prevent the tube 20 from moving during the overmoulding process. The skilled reader will appreciate that the mould 60 shown in FIG. 6 is by way of example only.

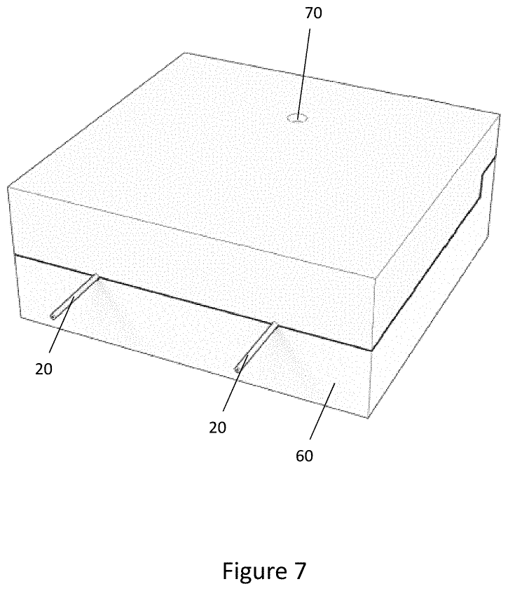

In step 503, when the mould 60 has been closed as shown in FIG. 7, the overmould material is injected into the mould 60 to form the overmould structure 30 in the encapsulation region overlapping the connector 22 and the tube 20. For example, the overmould material may be injected via the injection point 70 shown in FIG. 7. The overmould material then flows along the channels 64 to the overmould chamber 66 such that the liquid overmould material fills the overmould chamber 66 and encapsulates the parts of the connector 22 and the tube 20 that are within the overmould chamber 66.

The melt temperature of the overmould material is typically between about 110° C. and 130° C. The overmould material may be injected into the mould 60 at a temperature of between about 160° C. and 180° C. In an example moulding process, injection takes less than 0.5 seconds and the mould 60 is held together for around 4 or 5 seconds to allow the overmould structure 30 to begin to cool and cure. The mould 60 is then opened and the overmould structure 30 is allowed to cool for about 15 seconds in the mould 60 to allow the overmould structure 30 to cure and harden sufficiently to be removed from the mould. The skilled reader will appreciate that the aforementioned parameters are by way of example only and the parameters will vary depending on the application.

In step 504, after the overmould structure 30 is allowed to cool and at least partially cure, the connector 22 and tube 20 are removed from the mould 60 with the overmould structure 30 securing the connector 22 to the tube 20.

Many variations are possible within the inventive concept. For example, whilst the barb 44 has been exemplified as a circumferentially-extending retention feature in the figures, the barb 44 could instead comprise one or more radially extending tabs or a radially extending flange to define an engagement zone between the barb 44 and the proximal wall 46. Furthermore, the mechanical retention feature may comprise a proximally extending neck portion 43 without a barb 44. The neck 43 may comprise a series of ridges, ribs or indents that the overmould structure 30 may flow into thereby acting as a mechanical retention feature. Such features advantageously increase the surface area of the connector 22 to which the overmould 30 may bond to.

The skilled reader will appreciate that the barb 40 could be omitted if the heat-bond between the connector 22 and the overmould structure 30 is sufficient to secure the connector 22 to the tube 20 in certain medical applications. Alternatively, another shape of anchor formation could extend proximally from the connector body 42 around the tube 20 to extend the surface area of the interface between the overmould structure 30 and the connector 22. For example, such an anchor formation could comprise a plain or circumferentially-ridged sleeve around the tube 20.

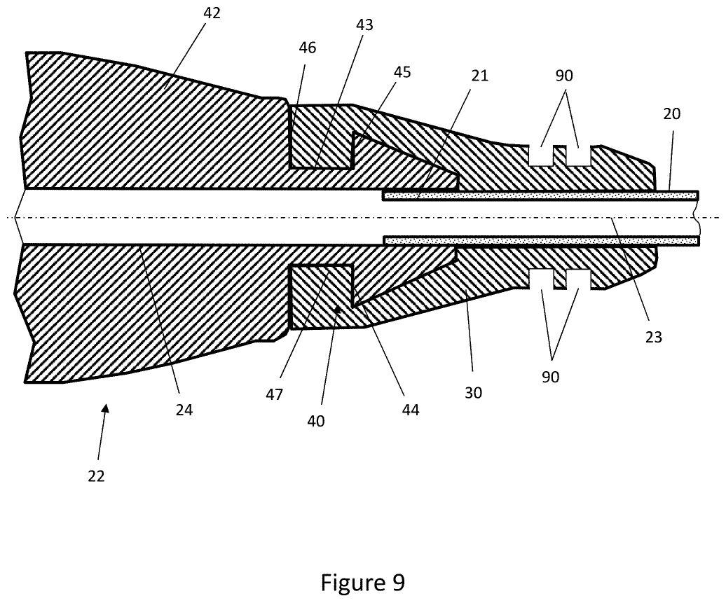

As shown in FIG. 9, the overmould structure 30 may comprise a series of circumferentially extending rings 90 on the outer surface of the overmould structure 30. The rings 90 beneficially remove material from the overmould 30 and thus increase the flexibility of the overmould structure 30. This improves the functionality of the overmould 30 in terms of acting as a strain relief. The addition of such rings 90 are particularly beneficial when the overmould structure 30 is made from a relatively rigid material that otherwise would not provide sufficient strain relief to the tube 20.

The skilled reader will appreciate that whilst the overmould structure 30 has been described in the context of overmoulding it may equally be formed from another manufacturing process, for example, via vacuum casting, compression moulding or through injecting a two-part resin that may encapsulate a portion of the tube 20 and the connector 22 to form the overmould structure 30.

It will be appreciated that various changes and modifications can be made to the present invention without departing from the scope of the claims.

1. A medical tube for delivering fluids to a patient, the tube comprising:
a flexible tubular body for conveying the fluids; and
a connector for connecting the medical tube to a source of fluids;
wherein the connector is secured to the tubular body by an overmould structure that encapsulates a portion of the tubular body and at least a portion of the connector thereby securing the connector to the tubular body.

2. A medical tube as claimed in claim 1, wherein the connector comprises a central bore and a distal end of the tubular body is received within the central bore.

3. A medical tube as claimed in claim 1, wherein the connector comprises a mechanical retention feature for securing the overmould structure to the connector.

4. A medical tube as claimed in claim 3, wherein the mechanical retention feature comprises a distally-facing wall and a proximally-facing wall that are mutually spaced in a longitudinal direction to define an annular recess between them.

5. A medical tube as claimed in claim 4, wherein a portion of the overmould structure extends into and engages within the annular recess.

6. A medical tube as claimed in claim 3, wherein the mechanical retention feature comprises a proximally-tapering barb.

7. A medical tube as claimed in claim 3, wherein the overmould structure encapsulates the mechanical retention feature.

8. A medical tube as claimed in claim 3, wherein the overmould structure tapers from a first diameter at a distal end to a second diameter at a proximal end.

9. A medical tube as claimed in claim 8, wherein the overmould structure extends proximally from the connector to a length that exceeds the first diameter.

10. A medical tube as claimed in claim 6, wherein an outer surface portion of the overmould structure tapers proximally and that tapered portion is longitudinally aligned with the proximally-tapering barb.

11. A medical tube as claimed in claim 1, wherein an outer surface portion of the overmould structure is substantially aligned with a longitudinal axis of the connector.

12. A medical tube as claimed in claim 1, wherein the overmould structure is bonded to the connector and to the tubular body.

13. A medical tube as claimed in claim 12, wherein the bond is a heat-bond and wherein the heat-bond forms a seal between the overmould structure and the connector and/or the tubular body.

14. A medical tube as claimed in claim 1, wherein the connector has a flexural modulus of at least 700 MPa and the tubular body has a flexural modulus of less than or equal to 100 MPa.

15. A medical tube as claimed in claim 1, wherein the overmould structure has a flexural modulus that is equal to or greater than a flexural modulus of the tubular body.

16. A medical tube as claimed in claim 1, wherein the overmould structure has a flexural modulus that is lower than or equal to a flexural modulus of the tubular body.

17. A medical tube as claimed in claim 1, wherein the overmould structure is moulded from an injection-mouldable version of the material of the tubular body.

18. A medical tube as claimed in claim 1, wherein the material of the overmould structure has a melting or softening temperature that is equal to or lower than a melting or softening temperature of the tubular body.

19. A medical tube as claimed in claim 1, wherein the material of the overmould structure has a melting or softening temperature that is equal to or higher than a melting or softening temperature of the tubular body.

20. A feeding tube for enteral feeding, the feeding tube comprising:
a flexible tubular body for conveying nutrients to a patient's stomach; and
a connector for connecting the flexible tubular body to a source of nutrients;
wherein the connector is secured to the tubular body by an overmould structure that encapsulates a portion of the tubular body and at least a portion of the connector thereby securing the connector to the tubular body.

21. A feeding tube as claimed in claim 19, wherein the feeding tube is connected to a syringe or a pump for delivering nutrients to a patient.

22. A method of manufacturing a medical tube for use in a medical application, the medical tube comprising a flexible tubular body and a connector, the method comprising:
placing an end portion of the tubular body inside the connector;
placing the tubular body and the connector into a mould; and
injecting an overmould material into the mould to encapsulate a portion of the tubular body and at least a portion of the connector, forming an overmould structure that secures the connector relative to the tubular body.

23. The method as claimed in claim 22, wherein the method comprises forming the connector prior to injecting the overmould material.
